(19) United States
(12) Patent Application Publication
(10) Pub. No.: US 2023/0313264 A1
(43) Pub. Date: Oct. 5, 2023

(54) ASSAY AND METHOD FOR MEASURING ALKALINE PHOSPHATASE ACTIVITY IN URINE AS AN INDEX OF TISSUE ZINC DEFICIENCY

(71) Applicant: VISION DIAGNOSTICS, INC., BRANFORD, FL (US)

(72) Inventor: JERRY W. DENNEY, BRANFORD, FL (US)

(21) Appl. No.: 18/011,545

(22) PCT Filed: Jul. 1, 2021
(86) PCT No.: PCT/US2021/040050

§ 371 Date: Dec. 20, 2022

Related U.S. Application Data
(60) Provisional application No. 63/046,764, filed on Jul. 1, 2020.

(51) Int. Cl. C12Q 1/42

(52) U.S. Cl. CPC C12Q 1/42

(57) ABSTRACT

A kit comprising two reagents for conducting two different Assays to measure a zinc-activated enzyme in a bodily fluid. The assay results are utilized to obtain a relative zinc-activated enzyme activity value that can be correlated to tissue zinc levels. A more specific embodiment utilizes two paired reagents for conducting an A Assay and a B Assay that measure different alkaline phosphatase (ALP) activities in a urine sample to obtain a Relative Renal Alkaline Phosphatase Activity (RRAPA) value. The RRAPA value can be correlated to zinc levels in renal tissue.

### CROSS-REFERENCE TO A RELATED APPLICATION

This application claims the benefit of U.S. Provisional Application Serial No. 63/046,764, filed Jul. 1, 2020, the disclosure of which is hereby incorporated by reference in its entirety, including all figures, tables and amino acid or nucleic acid sequences.

### BACKGROUND OF INVENTION

The Covid-19 virus pandemic focused attention on sectors of the population particularly vulnerable to the virus. While some infected persons exhibit mild or even unnoticed symptoms, other infected individuals progress rapidly to life threatening illness. The progression is thought to result from a rapidly produced high virus titer and the consequent cytokine storm created in the body that leads to deterioration of health or even death. There are currently several vaccines available, but there are still concerns about the virus. If all symptoms were mild, there would be no reason for fear. In fact, infection would be a natural form of vaccine.

It became apparent early in the pandemic that the elderly, diabetics, persons with cardiovascular disease, African ancestry, the morbidly obese, those in poverty, and other groups with co-morbidities were particularly vulnerable to adverse outcomes including death. One characteristic common to these most vulnerable individuals are high rates of zinc deficiency relative to the general population.

Climate may also have played a role. States along the U.S. Northern border had higher death rates than did Florida, Texas, and California. The pandemic struck the United States in February and March when the Northern U.S. was still in the grip of Winter, which kept people indoors or covered up when they ventured outside. In response to the increased infections, it was mandated that people stay indoors and avoid outside activities, thus, further insuring that people get less sun exposure. This reduced sun exposure resulted in lower serum levels of vitamin D. People in general received even less when they began wearing masks and remaining inside. Elderly patients in nursing homes received almost no sun.

Sun exposure on the skin causes the body to produce vitamin D. A major cause of adverse outcome in patients suffering from Covid-19 infection has been thrombotic events affecting the circulatory and pulmonary systems. To combat the effects, patients are treated with Heparin in an attempt to reduce coagulation. Unfortunately, Heparin can interfere with the bodies’ ability to utilize vitamin D. Combined deficiencies of vitamin D and zinc may be particularly lethal for infected persons. Vitamin D is believed to be necessary for the absorption and transport of zinc and is essential for the homeostasis of plasma and tissue zinc.

New viral diseases such as human-immunodeficiency (HIV), Severe Acute Respiratory Syndrome (SARS), and the new Covid-19 coronavirus have been shown to cause production of cytokines that trigger auto-immune disorders. There has been considerable interest and research conducted to try to obtain a more complete understanding of the immune system.

Zinc deficiency has a number of symptoms: loss of taste, loss of smell, weight loss, growth retardation, atrophy, immune dysfunction, increased oxidative stress, adversely boosted inflammatory response, skin abnormalities, hypogonadism, cognitive impairment, lymphopenia, decreased ratio of T helper cells to cytotoxic T cells, inadequate T cell differentiation, decreased natural killer cells, increased monocyte cytotoxicity, oligospermia, hyperammonemia, low cytokine production, and secretion of pro inflammatory IL 6 is pathologically elevated. Additionally, there is a negative influence on critical neutrophil functions: Phagocytosis, oxidative burst, degranulation, cytokine production, chemotaxis, neutrophil extracellular trap (NET) formation and apoptosis.

Despite, or perhaps because of, the array of clinical symptoms, zinc deficiency is currently recognized to be very difficult to diagnose clinically and diagnosis is likely to be delayed until severe and often irreversible damage to health has occurred.

At 2-3 g in total, zinc is the second most abundant metal in humans and is distributed unequally throughout different organs and tissues. Prostate, pancreas, and bone are considerably high in zinc, containing up to 200 µg/g. In contrast, zinc concentrations in heart, brain and plasma are comparatively low at 1-23 µg/g. Although plasma has only 1 µg/g, it is probably the most important reservoir for zinc homeostasis.

Taste-acuity testing has been used to detect tissue-zinc deficiency. Gustin is a zinc-containing protein in saliva that is thought to mediate taste acuity. Four tastes have been used for assessment: salt (NaCl), bitter (urea), sour (HCl) and sweet (sucrose). Obtaining valid taste accuracy requires considerable time and attention by both the subject and the test administrator. In an initial study, supplementation with zinc sulfate was found to benefit patients with idiopathic hypogustia. However, a later double-blind study showed no effect of zinc over placebo in improving taste acuity. There have been mixed results based upon the population tested. Obviously, taste acuity testing is not automated and large scale testing would be difficult.

Plasma zinc testing using atomic absorption or flame photometry has become the method of choice for testing for zinc. However, the U.S. Health and Human Services echoed the findings of many reports that plasma zinc levels are ineffective in detecting zinc deficiency due to the human body’s mechanism to maintain homeostasis of blood plasma zinc. These tests are further disadvantaged because they are not performed by high speed automation and most clinical laboratories do not have the needed equipment, thus making broad screening impractical.

In 2016, an Expert Panel of the American Society of Nutrition found, that zinc dependent enzymes have been used as biomarkers of zinc status in a number of studies. Those studies showed that enzymatic and blood cellular zinc biomarkers do not relate consistently to changes in zinc intakes or PSZs [plasma zinc concentration]. Thus, the Expert Panel classified these biomarkers as ‘not useful’. [J NUTR 2016; 146(Suppl):858S-885S [see page 874S, section “Biomarkers Not Recommended”]. The enzymes rejected as “not useful” by the Expert Panel included nucleotide polymerases, carbonic anhydrases, extracellular superoxide dismutase, aminolevulinic acid dehydratase, angiotensin converting enzyme, plasma #5 nucleotidase, and alkaline phosphatase (ALP).

The publication also stated that the lack of established cutoffs for evaluating zinc status and the need to collect a urine sample every hour for 24 hours obviates the practical usefulness of urinary zinc as a biomarker. However, the expert panel has said that examining individual isoenzymes may yield more sensitive and consistent data. [Page 875 S]. The ALP in urine is from kidney tissue and, thus, is the kidney ALP isoenzyme, not bone or intestinal isoenzyme found in serum.

Plasma zinc testing using atomic absorption or flame photometry has become the method of choice for testing for zinc. However, the U.S. Department of Health and Human Services echoed the findings of many reports that plasma zinc levels are ineffective in detecting zinc deficiency due to the human body’s mechanism to maintain homeostasis of blood plasma zinc. Plasma zinc has also been reported to be normal in certain patients with confirmed zinc deficiency and is now considered to be unreliable.

Urine zinc measurements have been used but are disadvantaged because they must be acquired at least hourly over a 24-hour period and an acid must be added to samples to prevent zinc precipitation. A potential problem in quantifying a urine analyte is the further fact that analyte excretion varies during the day based upon varying water intake. To correct for this variation, analytes are traditionally reported as the amount excreted per 24-hour period. For example, urine zinc is reported as the milligrams excreted per 24 hours. The amount of zinc excreted over the 24-hour period (usually only about 0.3-0.6 mg/day) can be calculated. The results, which may be helpful in determining plasma zinc levels, are not effective for detecting overall zinc deficiency.

These tests are further disadvantaged because they are not capable of being performed by high speed automation and most clinical laboratories do not have the necessary testing equipment that would make broad screening practical. Scientific research on various aspects of diseases caused by zinc deficiency is hampered due to the absence of a reliable test for zinc deficiency, and there is a broad consensus that a reliable bio-marker for zinc deficiency needs to be found.

Mass screening with a reliable and effective tissue-zinc deficiency test is needed to detect persons at high risk of severe damage to health or death due to a viral infection. A safe and effective treatment for zinc deficiency has been demonstrated on thousands of patients over multiple years within the study on AREDS 2™ for the treatment of AMD. There has been a recommendation that AREDS 2™ be investigated for potential prophylactic protection against Covid-19. In the absence of a reliable test for zinc deficiency, particularly at the tissue level, it must also be determined who would benefit from zinc supplementation.

The inability to conduct mass screening of patient samples for tissue-zinc deficiency means there is no way to quickly and accurately detect those individuals at high risk of severe damage to health or death due to a viral infection.

Although safe and effective treatment is available for zinc deficiency, there is no reliable procedure for detecting zinc deficiency. Recently, the world-wide medical community has called for the development of an assay that utilizes a biomarker to detect tissue-zinc deficiency.

### BRIEF SUMMARY

Embodiments of the subject invention provide a kit that comprises two paired-reagents that are utilized with two assays. The two paired-reagents have a substrate that can react with a biomarker commonly found in human urine that forms a product that can be accurately measured and the measurement correlated to zinc levels in body tissue. More specifically, the two paired-reagents can react with zinc-activated enzymes that derive from one or more body tissue. In a particular embodiment the reagents react with alkaline phosphatase (ALP) in urine. The kit can be used with a reliable and effective method for determining tissue zinc levels and, in particular, detecting tissue zinc deficiency by obtaining a relative activity value.

In one embodiment, the kit comprises two reagents that react with a zinc-activated tissue enzyme found in a bodily fluid, such as urine, plasma, spinal fluid, or saliva, to obtain a relative activity value, which can be referred to generally as a “Relative Zinc-Activated Enzyme Activity (RZAEA)” value. In a more specific embodiment, the kit comprises two paired-reagents that comprise a substrate that reacts with ALP in urine to produce a product that can be measured and used to obtain a relative activity value, referred to herein as a “Relative Renal Alkaline Phosphatase Activity (RRAPA) value,” from a single urine sample, from which a first portion and a second portion are obtained, which is one of the least invasive types of bodily fluid samples that be obtained. Advantageously, the two paired-reagents can be used with standard high-capacity automated clinical chemical analyzers that are currently used in professional laboratories to conduct mass screenings of urine samples.

Embodiments of the subject invention are described for utilizing alkaline phosphatase (ALP) as an effective biomarker. ALP is involved in several excretory and other zinc-related functions and kidney tissue comprises high levels of ALP and zinc, which are also reliably found in urine. ALP comprises two zinc atoms and production in the body is dependent on tissue zinc levels, so is not produced in the absence of sufficient zinc. Thus, the amount of ALP in tissue can be, advantageously correlated to tissue zinc levels and in some embodiments can at least indicate a zinc deficiency. Embodiments of the subject invention provide a test methodology that utilizes urine for measuring the native activity of renal-derived ALP and correlating the result to the homeostatic status of tissue zinc. The test methodology can also be applied to other types of bodily fluids, such as plasma, saliva, and spinal fluid, for measuring the native activity of other tissue-derived zinc-activated enzymes and correlating the results to the homeostatic status of tissue zinc.

The average urine zinc concentration for an individual has been determined to be about 34% of the mean blood plasma level for the same individual. The lower urine zinc level may be due to the fact that most of the zinc in blood plasma is bound to a variety of proteins found in plasma, including transferrin, albumin, and others. Proteins are not normally present in the glomerular filtrate, so that only zinc associated with smaller molecules, involved in targeted transport, would typically be found in urine. The relatively low level of zinc in urine as compared to plasma is a desirable characteristic exploited by the embodiments of the subject invention.

Embodiments of the subject invention utilize two paired reagents with an advantageous single, randomly acquired urine sample from an individual. From the single urine sample an amount is used as a first portion and another amount is used for a second portion. The subject invention provides an inventive solution to the varying excretion dilution problem by utilizing an internal standard “A Test” (with optimal zinc) used with the first portion of the urine sample and producing full activity of the renal ALP in combination with a “B Test” (zinc-free reagent) used with the second portion of the urine sample, which gives the native activity of the renal ALP. The percent activity, referred to herein as the Relative Renal Alkaline Phosphatase Activity (RRAPA), can be obtained by dividing the measurement obtained with the B Test (native renal ALP) by the optimally zinc-activated A Test. Thus, embodiments of the subject invention can detect the relative activity of renal-tissue alkaline phosphatase (ALP) as a biomarker for zinc deficiency and provide a Relative Renal Alkaline Phosphatase Activity (RRAPA) percentage value. This is different from previous efforts to determine the amount of ALP during a 24-hour period, which were devised to determine zinc intake of an individual.

The concentration of ALP in a given urine sample can vary during any given period due to activities of the individual. For example, if a person drinks water some time before providing a urine sample, the sample can be more diluted than it would have been if provided beforehand. Thus, measuring ALP in the urine sample at any given time can produce different results, which may lead to an incorrect assumption about tissue zinc levels. The assays of the subject invention, by utilizing two paired-reagents, resolve the problem of varying dilution in urine samples, which causes correspondingly varied ALP levels in the urine. The first test A and associated Reagent I is designed to determine the level of total ALP in the sample. The second test B and associated Reagent II is designed to determine the activity of the kidney-tissue related or renal-derived ALP in the urine. The results of each test can be measured by spectrophotometric techniques know in the art and used by standard high-capacity automated clinical chemical analyzers found in most laboratories and facilities that perform mass sample screenings.

There is some tendency for renal-zinc deficient specimens to have ALP levels that are low. This is believed to be due to the constitutive role of tissue zinc in the formation of renal ALP. Embodiments of the subject invention can utilize a weighting factor to adjust for the effect of a urine sample with a low amount of ALP. The reagents of the subject invention can advantageously be used with automated clinical chemistry analyzers to perform. Such equipment is typically calibrated prior to conducting screenings with a particular reagent. Because each of A Assay and B Assay utilize a different reagent, calibration errors can affect results. To counter this effect a Numeric Factor can be utilized when calculating the RRAPA.

Urine samples can also have a variety of contaminants such as, for example, erythrocytes, cellular components, bacteria, and other non-biological contaminants. To reduce the effect of these contaminants, the urine sample can be centrifuged prior to conducting the tests.

### BRIEF DESCRIPTION OF DRAWINGS

FIG. 1 illustrates the stages of viral replication cycles that can be inhibited by the presence of zinc. In vitro studies have shown that zinc interferes with a number of mechanisms of viral replication cycles, including free virus inactivation. Some of the cycles that are interrupted or halted by zinc are inhibition of viral uncoating (1); viral genome transcription (2); viral protein translation and polyprotein processing (3). There have been no studies that demonstrate zinc-medicated inhibition of virus assembly and/or particle release (4). Abbreviations: CV, coronavirus; DdDp, DNA-dependent DNA polymerase; EMCV, encephalomyocarditis virus; FMDV, foot and mouth diseases virus; HCV, hepatitis C virus; HIV, human immunodeficiency virus; HPV, human papilloma virus; HRV, human rhinovirus; HSV, herpes simplex virus; PV, polio virus; RdRp, RNA-dependent RNA polymerase; RT, reverse transcriptase; SARS severe acute respiratory syndrome coronavirus; SFV, Semliki Forest virus; SV, sindbis virus; VZV, varicell-zoster virus; Zn, zinc.

FIG. 2 is a graph that demonstrates the RRAPA results obtained in an elder population study, discussed in Example 2.

FIG. 3 is a graph that demonstrates the zinc measurements obtained from random urine samples provided by an 89-year old volunteer. The random urine samples from the volunteer were obtained over a period of 14 days. The volunteer initially tested with a low zinc level, as shown in the graph. The volunteer was subsequently administered a daily dose of AREDS 2™ (Bausch & Lomb), which the graph shows resulted in an increase in tissue zinc level indicated by the RRAPA measured in the urine samples.

FIGS. 4A and 4B are graphs that demonstrate the viability of the embodiments of the two paired reagent test, according to the subject invention, for measuring the activity of ALP in renal-tissue of an individual. FIG. 4A illustrates the amounts of creatinine and ALP measured in urine samples from 100 individuals. Creatinine concentration is a function of the dilution of urine, which can be caused by a variety of factors, including fluid consumption. As shown in FIG. 4A there is minimal correlation between the creatinine and ALP in the individuals tested. FIG. 4B illustrates the amounts of creatinine correlated to the ALP% activity utilizing embodiments of the A Assay and the B Assay with the reagents of the subject invention. As shown in FIG. 4B, the embodiments of the subject invention provide an accurate ALP% activity in the renal tissue of each individual that is independent of the creatinine levels. This indicates that embodiments of the methods and reagent of the subject invention are not affected by dilution of the urine. Note: these results are based on uncentrifuged urine samples and were not inspected blood in the samples.

FIGS. 5A and 5B are screen shots taken of settings that can be programmed into a Beckman Coulter AU400 series automated clinical chemical analyzer when utilizing an embodiment of the kit of the subject invention for obtaining a RRAPA of a urine sample.

FIGS. 6A and 6B are graphs that illustrate one embodiment of the method for utilizing the two paired tests of the subject invention to analyze a urine sample with an automated clinical chemistry analyzer.

FIG. 7 is a graph of the RRAPA results calculated from the data in FIGS. 6A and 6B.

FIG. 8 is a graph of random urine collections (centrifuged) that show the RRAPA results are unaffected by urine concentration/dilution of the sample.

FIG. 9 represents data from 51 individuals that provided a urine sample (centrifuged) that was utilized with the kit and assays of subject invention to obtain a RRAPA. The RRAPA results were used to establish that a low cutoff level for tissue zinc is approximately 42%, indicative of a zinc deficiency.

FIGS. 10A and 10B are graphs of the data shown in FIG. 9 broken down into the gender (10A) and age (10B) of the individuals. In both 11A and 11B graphs, the samples were centrifuged.

FIG. 11 is a graph of the data presented in Table 2.

FIG. 12 is graph of the results obtained by controlling zinc intake or depletion a 50-year old male volunteer.

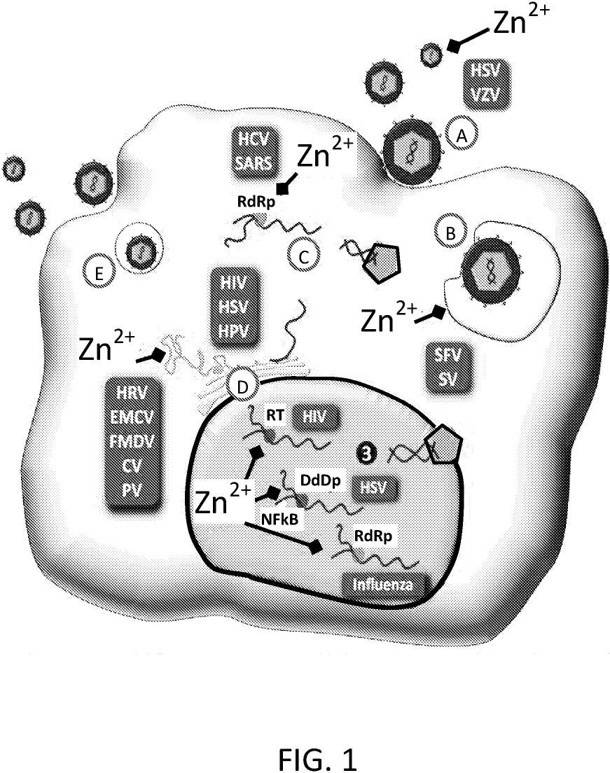

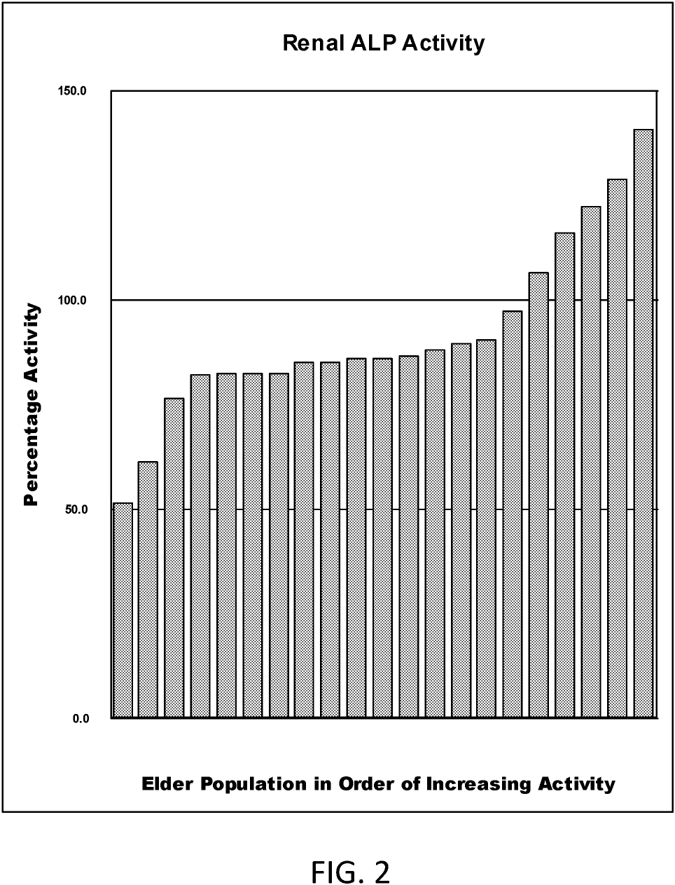

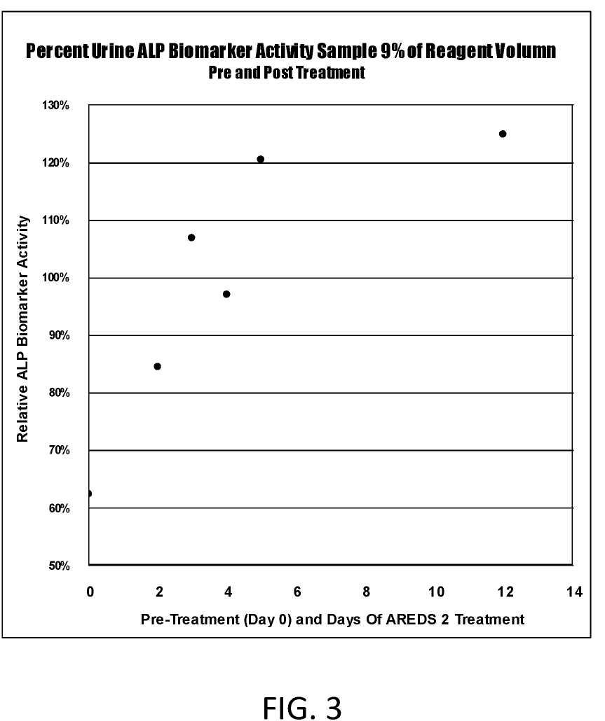

### DETAILED DISCLOSURE

Embodiments of the subject invention pertain to an kit that utilizes two paired reagents in a two test methodology for measuring alkaline phosphatase (ALP) in a urine sample to obtain a Relative Renal Alkaline Phosphatase Activity (RRAPA) value, which can be used as an indicator of the zinc level/deficiency in tissue. Embodiments of the subject invention include an A Assay that uses the paired Reagent I, for obtaining a measure of the amount of total ALP in a urine sample by measuring the amount of product formed by the ALP reacting with a substrate in the Reagent I and a B Assay that uses a Reagent II and is used to obtain a measure of the activity of the ALP in the urine sample by measuring the amount of product formed by the ALP reacting with the same substrate as is used in Reagent I. The results obtained from each Assay are utilized in the RRAPA Equation to calculate a RRAPA Value.

While the subject application describes embodiments of a kit and method for obtaining a relative activity value for alkaline phosphatase derived from renal tissue, referred to herein as a “Relative Renal Alkaline Phosphatase Activity (RRAPA)” value, the embodiments of the subject invention are not limited to use with alkaline phosphatase or urine. The embodiments of the subject invention could be utilized to obtain a relative activity value for other zinc-activated enzymes derived from other bodily tissue and measured in other bodily fluids, such as, for example, plasma, spinal fluid, or saliva. There are numerous tissue-derived zinc-activated enzymes found in urine, plasma, spinal fluid, and saliva. Many can be detected by various techniques and reagents known in the art, but have previously been considered ineffective as indicators of tissue zinc levels. This is because previous methods focused on measuring only the amount of the enzyme in a bodily fluid sample, which studies have shown does not accurately correlate to tissue zinc levels. The subject invention describes how two reagents can be used to obtain different measurements of the zinc-activated enzyme (total enzyme activity and native enzyme activity) with a method for calculating a relative activity value using the resulting two different measurements of the zinc-activated enzyme to obtain a relative value that can be more generally referred to as a Relative Zinc-Activated Enzyme Activity (RZAEA) value. Thus, a RZAEA can be calculated for any zinc-activated enzyme and that value can be more accurately correlated to tissue zinc levels.

In the first ALP enzyme measurement obtained with the A Assay, a first paired Reagent I was formulated based on the procedures set forth by the IFCC Standard Method, for a reagent represented to provide optimal zinc activation for the ALP assay. For the second measurement obtained with a second B Assay, embodiments of the subject invention utilize a novel paired Reagent II. Reagent II is identical to Reagent I, which is based on the IFCC method, but does not include zinc sulfate, which is present in Reagent I.

In the development of the A Assay and the B Assay and the two paired reagents used with each one, consideration was given to the likelihood that the renal-tissue derived ALP is associated with cell membranes and therefore associated with lipid. As renal tissue derived ALP enters the urine, activity may be partially blocked by such lipid. Consequently, non-ionic detergents were tested including Brij 35 and Triton-X 100. It was found that both increased activity, with the A Assay reaction being the most improved and Brij 35 being the most effective at increasing activity. Activity was also noticed to be slightly greater in samples a few days old. In one embodiment, the Reagents I and II of the subject invention depart from IFCC Standard Method Regent by inclusion of 0.1% w/v of Brij 35 in both reagents.

N-hydroxyethyl)-ethylenediaminetriacetic acid (HEDTA) is a zinc binding substance included in the IFCC ALP Standard Method Reagent to prevent precipitation of zinc. Embodiments of the subject invention include N-hydroxyethyl)-ethylenediaminetriacetic acid in both the Reagent I and Reagent II. This can ensure that the only difference between the two paired Reagents that would affect results is the presence of zinc sulfate in Reagent I.

The RRAPA value can be obtained by using the results of the A Assay and the B Assay in the following RRAPA Equation:

$$\text{RRAPA Value=} \left(\text{Results of B Assay} / \text{Results of A Assay}\right) * 100$$

In a more general embodiment, where a different zinc-activated body-tissue derived enzyme is used, a Relative Zinc-Activated Enzyme Activity (RZAEA) value can be obtained by using the results of an A Assay and a B Assay. The A Assay and B Assay can be specific to the chosen zinc-activated enzyme and the measurements obtained used in a similar equation:

$$\text{RZAEA Value} = \left(\text{Results of B Assay} / \text{Results of A Assay}\right) * 100$$

When measuring ALP, the RRAPA percent value represents the percent activity of the ALP bio-marker in the renal tissue. The ALP activity obtained with the A Assay can serve as an internal standard for test B, making the RRAPA percent value independent or unaffected by concentration/dilution of the urine sample. Advantageously, this improves accuracy and validity of the A and B assays, without the disadvantage of obtaining hourly samples over a 24-hour period.

One embodiment of the composition of the two paired Reagents I and II are shown in the following Table 1:

| | | | |
|---|---|---|---|
| A Assay--Reagent I Ingredients | Concentration* | B Assay--Reagent II Ingredients | Concentration* |
| 2-amino-2-methyl-1-propanol (buffer pH 10.4) | 0.35 mol/L | 2-amino-2-methyl-1-propanol (buffer pH 10.4) | 0.35 mol/L |
| Brij 35 non-ionic detergent | 0.1% w/v | Brij 35 non-ionic detergent | 0.1% w/v |
| 4-Nitrophenyl phosphate (sub strate/Indicator) | 16.0 mmol/1 | 4-Nitrophenyl phosphate (sub strate/Indi cator) | 16.0 mmol/l |
| Magnesium Acetate (activator) | 2.0 mmol/L | Magnesium Acetate (activator) | 2.0 mmol/L |
| N-hydroxyethylethylene-diaminetriacetic acid (HEDTA) | 2.0 mmol/L | N-hydroxyethylethylene-diaminetriacetic acid (HEDTA) | 2.0 mmol/L |
| Zinc sulfate (Activator) | 1.0 mmol/L | Zinc sulfate (Activator) | NONE |

* The concentrations are final concentrations of the reagents.

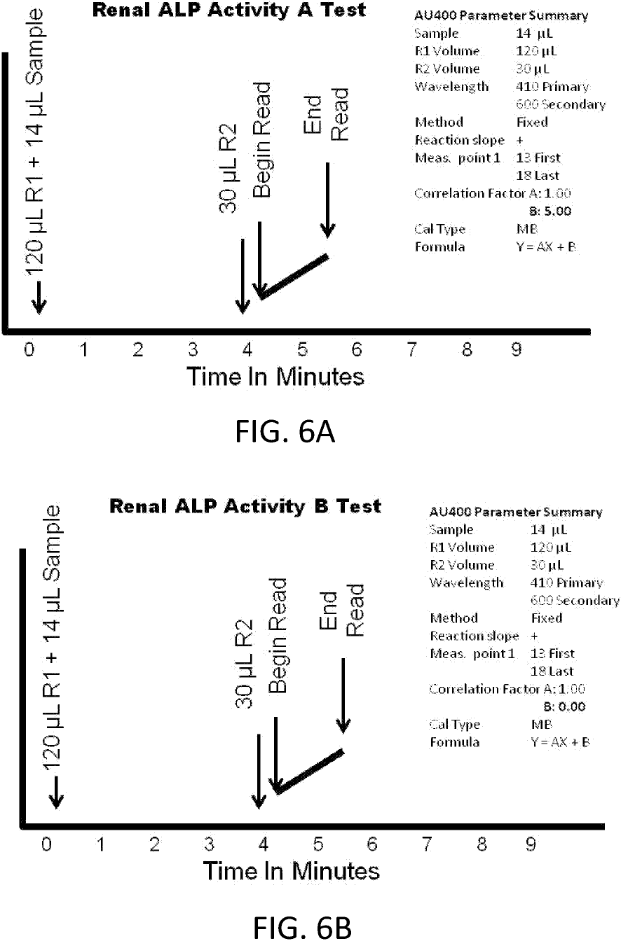

In a further embodiment, the two paired reagents each comprise a non-reactive sub-reagent and a reactive sub-reagent that includes the substrate 4-nitrophenelphosphate substrate. In a further embodiment, the second sub-reagent is more concentrated than the first sub-reagent, such that less of the second sub-reagent must be added to obtain the desired final concentration of the reagent. The A and B Assays can be performed by first adding the non-reactive sub-reagent to a first portion and second portion, respectively, of the respective urine sample, waiting for a time. This can allow the combined portions of the urine sample and non-reactive sub-reagent to warm-up. The reactive sub-reagent, comprising 4-nitrophenel phosphate is then added to the warmed-up first portion and second portion, respectively, of the urine sample and non-reactive sub-reagent to form a reactive urine sample. Once the reactive component is added, spectrophotometric analysis can begin to measure the product formed by reaction of the biomarker with the substrate. At the end of a pre-determined time the final measurement, which corresponds to the amount of yellow 4-nitrophenol product formed in the first portion and the second portion, respectively, of the urine sample, can be taken. FIGS. 6A and 6B illustrate non-limiting examples of one protocol for conducting the A Assay and the B Assay with an automated clinical chemistry analyzer.

In a more specific embodiment, the non-reactive sub-reagent of Reagent I, used with A Assay, contains an AMP buffered solution at pH 10.4 and 0.625 mmolar of Zinc with non-reactive preservatives and 0.02% Proclin^{®} 300 as a preservative. The non-reactive sub-reagent of Reagent II, used with the B Assay, has the identical composition as the non-reactive sub-reagent of Reagent I, except for the exclusion of zinc. The reactive sub-component of both Reagent I and Reagent II contain 1% w/v p-nitrophenolphosphate Chromogen with non-reactive preservatives and 0.02% Proclin^{®} 300 as a preservative.

The formation in the reactive urine sample of 4-nitrophenol from 4-nitrophenyl phosphate with Alkaline Phosphatase proceeds as shown in the following reaction:

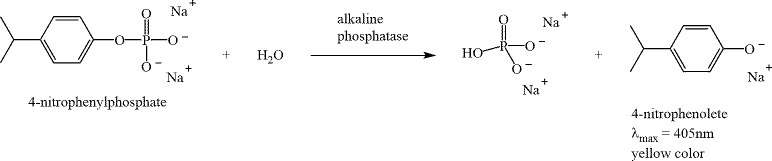

There are a number of instrument variables that can affect the measurement accuracy of any automated clinical chemistry analyzer, which can also affect the results of ALP measurements obtained with the tests and reagents of the subject invention. These include temperature, sampling and metering accuracy, pH differences, and others known to those with skill in the art. As a consequence, manufacturers of reagents often provide a reference serum for calibration, which adjusts for these variables.

Because the embodiments of the subject invention utilize two paired reagents with two different compositions, calibration of an automated clinical chemical analyzer, such as those typically used in laboratories that conduct large scale urine screenings, can bias the RRAPA calculations because of errors in the measurements due to variation in the accuracy of calibration between the two tests. In other words, the clinical chemistry analyzer can have a different level of calibration accuracy for each Assay, which can result in an inaccurate RRAPA results when the A Assay and B Assay results are utilized in the RRAPA equation.

In one embodiment, Factor Calibration is used when reporting results for both the Assay A and Assay B in order to eliminate variability in the RRAPA value due to imprecision of reference material calibration for the two paired reagents. Because the results obtained from the A Assay and B Assay are used to calculate the RRAPA, the Factor Calibration used can vary widely. This variation has no significant effect on the resulting RRAPA value, provided the Factor Calibration used is the same for the A Assay and the B Assay for any given sample.

Typically, the sample to reagent ratio of enzyme activity tests is chosen to be the largest practical sample amount, which produces the highest precision of measurement and allows the range of linearity to be sufficient for the range of activity in healthy and diseased samples. As such, because the amount of ALP in urine can be small, testing was started with a large sample: reagent ratio. In one embodiment, the sample volume comprises about 17% of the total reagent volume. The Reagent II contains HEDTA to complex urine zinc, but can be only partially effective with some application parameters. In particular, if the sample to reagent ratio has a high percentage of urine to reagent, the ALP activity can be elevated.

Further embodiments of the subject invention can provide more accurate results for native renal tissue derived ALP utilizing a smaller sample volume of urine to the total reagent volume. In one embodiment, the urine sample volume comprises about 1-2% of the total reagent volume. This can be optimal in preventing unwanted activation by urine zinc. In an alternative embodiment, to optimize for both precision (repeatability) and accuracy (freedom from minimal zinc interference) a urine sample volume is about 9% of the total reagent volume.

The amount of ALP is historically known to be unreliable in detecting zinc deficiency. It has observed that there is some tendency for renal-zinc deficient specimens to have low ALP levels, which is believed to be due to the constitutive role of tissue zinc in the formation of renal ALP. Thus, a low tissue zinc level will limit formation of ALP. In one embodiment, a weighting factor is utilized when calculating the RRAPA. The parameters used for both the A Assay and the B Assay can be the same except that a weighting factor is included in the A Assay calculation. In a further embodiment, this weighting factor is programmed into the automated clinical chemistry analyzer and used when calculating the results for the A Assay. In a specific embodiment, the A Assay calculation uses a weighting factor of +5 and the B Assay calculation uses a weighting factor of 0.

A urine sample can also contain numerous contaminants introduced by a variety of factors. Some of these contaminants can contain zinc or ALP that can affect the final Percent Activity. For example, non-renal ALP is present in vaginal epithelial cells, erythrocytes, leucocytes, organisms like trichomonas vaginalis, and other non-renal cellular elements. Erythrocytes are a particular concern as they contain 10X the level of zinc compared to plasma, which could mask abnormally low RRAPA.

Centrifugation of samples can be effective in eliminating contaminates that affect results when utilizing embodiments of the kit and assays of the subject invention. In one embodiment, urine samples are centrifuged prior to analysis and the supernatant is extracted and used with kit to conduct both the A Assay and the B Assay. Any centrifuged samples where the supernatant exhibits a red or pink color should be rejected for analysis, as these samples could contain erythrocytes.

Results obtained from a urine sample analyzed with embodiments of the kit of the subject invention with the A Assay and the B Assay are utilized in a RRAPA Equation to obtain a RRAPA value. The RRAPA value is reported as a percentage that indicates the activity of ALP in renal tissue, which can be correlated to the amount of zinc that was present in the renal tissue of the individual. A higher RRAPA value indicates greater activity of ALP in the renal tissue, which indicates a higher level of zinc in the tissue of an individual. Likewise, a lower RRAPA value indicates that the individual has a lower tissue zinc level. A RRAPA value below 42% can be an indication that the individual is suffering from a zinc deficiency. Since it has been shown that tissue zinc level has an effect on the robustness of the immune system, measuring the bio-availability of zinc can provide an indication of whether an individual has a greater likelihood of acquiring and combating viruses and other microbes.

The RRAPA value can be indicative of tissue zinc level. Studies conducted with embodiments of the reagents and methodology of the subject invention indicate that a RRAPA value of about 70.5% can be indicative of a normal tissue zinc level. A RRAPA of less than about 42% can be a low-end cut-off value and indicative of tissue-zinc deficiency. Graphical representation of the data is shown in FIG. 9. Following are examples that illustrate procedures for practicing the subject invention. These examples are provided for the purpose of illustration only and should not be construed as limiting. Thus, any and all variations that become evident as a result of the teachings herein or from the following examples are contemplated to be within the scope of the present invention.

### EXAMPLE 1: Procedure for Obtaining a Relative Renal Alkaline Phosphatase Activity (RRAPA) Percent Value with an Automated Clinical Chemistry Analyzer

Automated clinical chemistry analyzers are programmed with parameters or settings specific for the reagent and screening methodology to be performed. The parameters determine how the results will be interpreted and reported. FIGS. 6A and 6B include non-limiting examples of a Parameter Summary detailing settings that be utilized when programming a Beckman Coulter AU400 Series automated clinical chemical analyzer to perform a urinalysis with an A Assay and a B Assay with the kit of the subject invention.

In this methodology, Reagent I, in the kit, comprises sub-reagent 1 and sub-reagent 2, designated R1 and R2, respectively, in FIG. 6A. Before conducting the Assays, the urine sample was centrifuged and a first portion and a second portion of the supernatant was extracted for use. In the A Assay, the first portion can be combined with sub-reagent 1 (R1) and allowed to warm-up for approximately 4 minutes before sub-reagent 2 (R2) is also added to the first portion to complete Reagent I in the sample. After approximately 5.5 minutes, the urine sample is analyzed spectrophotometrically at a primary wavelength of 410 nm to measure the final amount of N-nitrophenol formed in the first portion of the urine sample. As shown in the Parameter Summary in FIG. 6A, the calculations include a weighting factor of 5, which is referred to as a Correlation Factor B: on the Parameter Summary. FIG. 6B shows that the same procedure is followed for the B Assay. As indicated in the Parameter Summary in FIG. 6B, the weighting factor is 0 for the B Assay.

EXAMPLE 2: Steps for Configuring Beckman Coulter AU 400 and AU640 Automated Clinical Chemical Analyzers for Measuring Alkaline Phosphatase in a Urine Sample with the A and B Assays to obtain a RRAPA value
1. Select Parameters - Common Test Parameters - Test Name
2. Find open location hit SET then EDIT type the name Z ACT in available location.
3. Select Long Name from tab at top then under Long Name type Z ACT Then hit EXIT
4. Select Parameters - Interrelated Tests- Calculated Tests then hit Set
5. Select Parameter then Common Test Parameters then select Calculated Test Tab then hit set and select the test to be calculated.
6. Under Test Name A hit pull Down and select Z DEF A
7. Under B Pull down Select Z DEF B
8. Under Constant a type 100.00
9. Under Formula type B/A *a
10. Exit
11. To set up ranges select Specific Test Parameters
12. Select Range tab
13. Use drop down to select Z ACT Test
14. Hit Set
15. Set ranges at for Low and for High
16. Select Exit
NOTE: Z ACT will not appear on the screen report but will be printed out automatically.

### EXAMPLE 3: Validation of Relative Renal Alkaline Phosphatase Activity (RRAPA) Assay

Plasma and serum zinc levels had been the most common method utilized for tests in published studies to determine zinc status. A Panel of Experts from the American Society of Nutrition and the National Institutes of Health (NIH), have concluded that these tests can be unreliable because of the homeostatic regulation of serum and plasma zinc. Specifically, the NIH Office of dietary supplements in Zinc: Fact Sheet for Health Professionals states that: “Plasma or serum zinc levels are the most commonly used indices for evaluating zinc deficiency, but these levels do not necessarily reflect cellular zinc status due to tight homeostatic control mechanisms. Clinical effects of zinc deficiency can be present in the absence of abnormal laboratory indices.” Cited reference: Maret W., Sandstead H.H. Zinc requirements and the risks and benefits of zinc supplementation. J Trace Elem Med Biol 2006; 20:3-18.

The Expert Panel review gave two reasons which “obviate the usefulness” of the tests utilized in these predicate studies of Urine Zinc measurements for the evaluation of zinc status:
A: The need for hourly collection of urine over 24-hours, and
B: Difficulty in establishing cutoffs using Zinc as the biomarker.

Published studies illustrate the problem with the determining the cutoffs utilized in these predicate studies:

| | | |
|---|---|---|
| Henry (1974) reported a range of: | 150-1,300 | µg zinc per 24 hrs |
| Tietz (1983) reported a range of: | 150-1,000 | µg zinc per 24 hrs |
| Am. Soc. of Nutrition reported a range of: | 300-600 | µg zinc per 24 hrs |

The following protocols establish that embodiments of the Relative Renal Alkaline Phosphatase Activity (RRAPA) assay of the subject invention is a safe and effective tool for the detection of tissue zinc status using random urine collection when used in accordance with the Instructions For Use (IFU). As used in this Example, the term “RRAPA assay” refers to a kit comprising a Reagent I and Reagent II, each comprising a reactive sub-reagent and non-reactive sub-reagent, utilized in an A Assay and a B Assay, which have been described in detail above.

Protocol A: Embodiments of the RRAPA assay address the need for a 24 hour collection by a test protocol demonstrating effective use of a single urine collection as commonly used for routine urinalysis.

Protocol B: Embodiments of the RRAPA assay address the problem with Predicate studies’ cutoffs with a protocol to compare two sets of 100 specimens each collected during two periods. The specimens were not centrifuged in order to define the magnitude of cellular contamination. The next part of the protocol used centrifuges specimens which were statistically analyzed for RRAPA data from 51 specimens collected for urinalysis and analyzed by the RRAPA assay.

As determined by the Expert Panel, previous studies using serum or plasma Alkaline Phosphatase (ALP) as a biomarker of tissue-zinc status are “not useful”. The principal ALP content of Serum is due to intestinal and bone ALP. In Predicate studies’ detection methods, the total amount of ALP was measured in zinc free reagents. However, the Expert Panel has said that examining individual isoenzymes may yield more sensitive and consistent data. The ALP in urine is derived from kidney tissue and, thus, is the kidney isoenzyme, which is different from bone or intestinal isoenzyme present in serum and plasma.

The Expert Panel has defined two key indicators required for the utility of a biomarker of zinc-status, which have been addressed in this study:
1. Measure the biomarker response after controlled manipulations of zinc intake, including both zinc-depletion/replication studies and zinc supplementation.
2. Compare biomarker level between individuals by using clinical signs that are generally recognized as functional outcomes of severe zinc deficiency.

Protocol C: Is designed to measure the Relative Renal ALP Activity (RRAPA) assay as a biomarker response after controlled manipulations of zinc intake, including both zinc-depletion/replication studies and zinc supplementation.

Protocol D: Is designed to compare the RRALPA assay biomarker level between individuals by using clinical signs that are generally recognized as functional outcomes of severe zinc deficiency.

Protocol E: Is designed to test the effectiveness of centrifuging urine specimens to eliminate contaminating cellular contamination.

### General Performance Characteristics

The automated assay on the Beckman Coulter AU400 automated clinical chemistry analyzer precision within-run and day-to-day and total imprecision were determined at normal (high) and abnormal (low) levels.

Precision Acceptance Criteria based upon published performance of the Predicate studies, which indicates a Coefficient of Variation < 13%. (A C.V. of 13% was published by Henry (1974)). The RRAPA assay was conducted with a 10 sample within run, 5 samples per day for 5 days for between run imprecision:

### Results

| Low (abnormal) sample: RRAPA of 45.8% | Coefficient of Variation | |
|---|---|---|
| | Within Run | 10.0% |
| | Day-to-Day | 0.62% |
| | Total Imprecision | 10.6% |
| High (normal) sample: RRAPA of 80% | Coefficient of Variation | |
| | Within Run | 3.0% |
| | Day-to-Day | 0.34% |
| | Total Imprecision | 3.34% |

Conclusion: The Precision of the RRAPA test is acceptable.

Carryover - The automated assay on the AU400 automated biochemistry analyzer was assessed for freedom from carryover.

### Results

No carryover was observed upon inspection of the carry over study.

### Linearity

Acceptable linearity was set as an r value greater than 0.900.

Water, low cutoff-level control, high control and an equal mixture of the low and high controls were assayed with five replicates each. Regression analysis was performed on the resulting RRAPA percent activities produced.

Result: r = 0.983. We concluded that linearity was acceptable.

### Stability

The Reagents I and II utilized in Tests A and B, respectively, of the RRAPA assay system have been shown to have a real-time shelf life of two years. In order to test minor variation in concentrations (principally zinc concentrations), the I reagents were subjected to accelerated life testing in accordance with industry established protocols, which are based upon the Arrhenius formula and experience with high energy compounds with temperature thresholds for stability.

### Results:

Subject two paired reagents were confirmed to be stable for two years when stored at 2-8° C.

High and low controls were tested for stability by either real time or accelerated life testing confirming the following:

| | |
|---|---|
| High Control: 2 years @ 2-8° C. (Real time) | 2 years |
| Low Control: 2 years @ 2-8° C. (Accelerated) | 2 years |

### Sensitivity:

The NIH experts stated the Predicate studies’ urine zinc test were insufficiently sensitive citing experimental studies showing that urinary zinc levels do not decline unless the dietary zinc intake is very low (<3 mg/day). (King, J.C., et al., page 872S).

The analysis of data from Protocol C, below, indicates that a 5.6% increase in the RRAPA assay value was achieved per each 5 mg/day of zinc supplementation. This indicates that the method of determine tissue zinc levels utilizing the RRAPA assay results has sufficient sensitivity.

Test Protocol A was designed to determine effectiveness of the RRAPA test with use of a single specimen collection of urine as is submitted for routine urinalysis as opposed to a 24 hour collection procedures utilized in Predicate studies. Two hundred urine specimens collected for routine urinalysis were obtained following the testing for which they were collected. The specimens had been refrigerated between 2 to 8 degrees centigrade since collection, but contained no antimicrobial preservative and were not inspected for visible blood. The un-centrifuged specimens were assayed with the RRAPA paired Reagent I for the A Assay (optimal zinc) and Reagent II for the B Assay (zinc free).

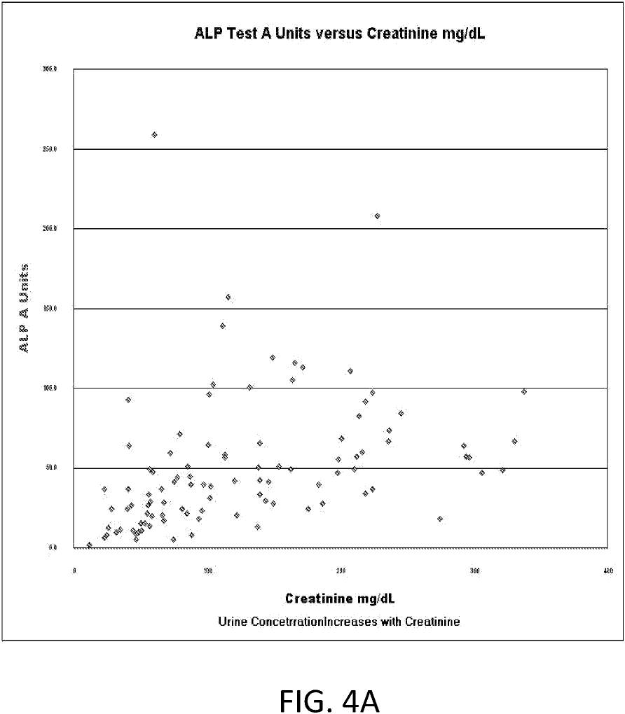

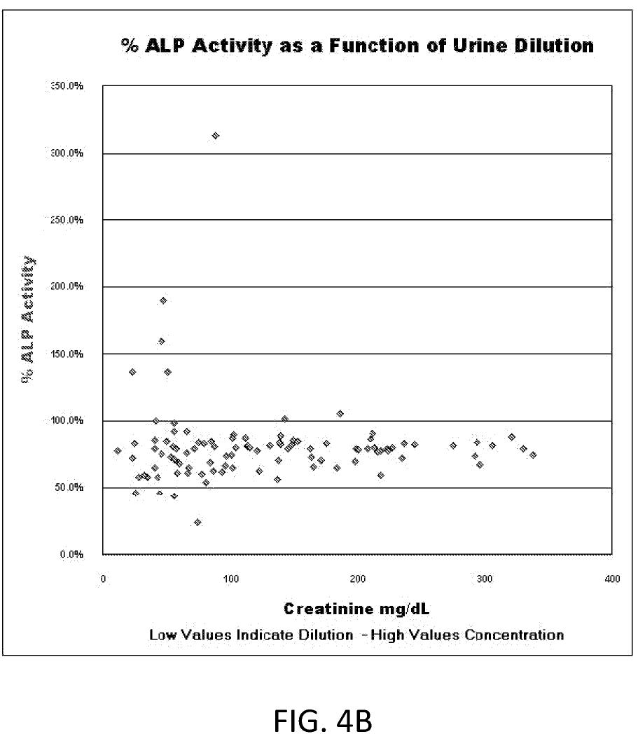

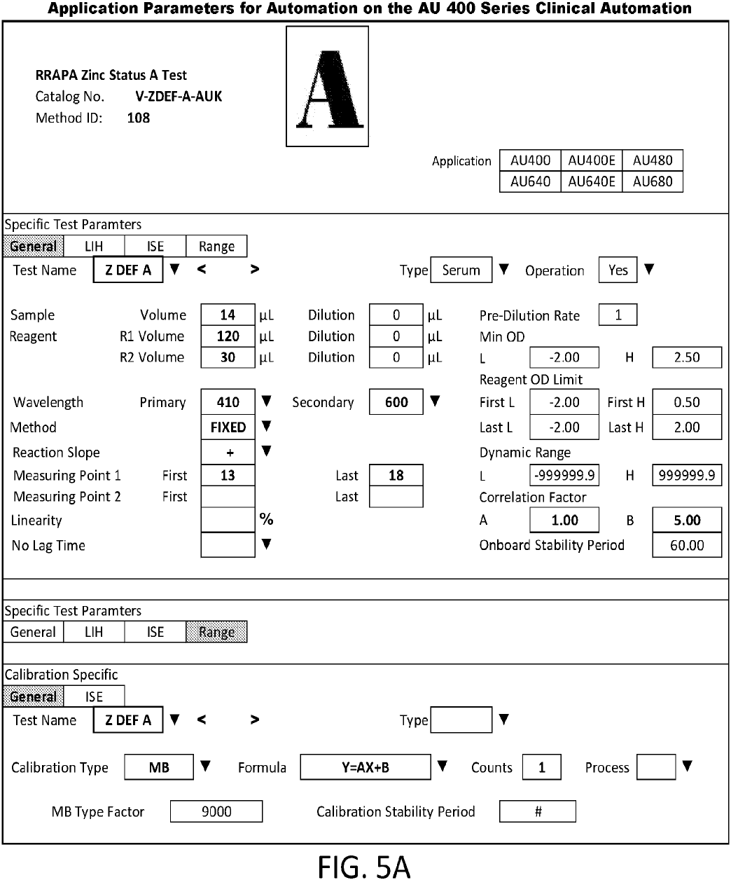

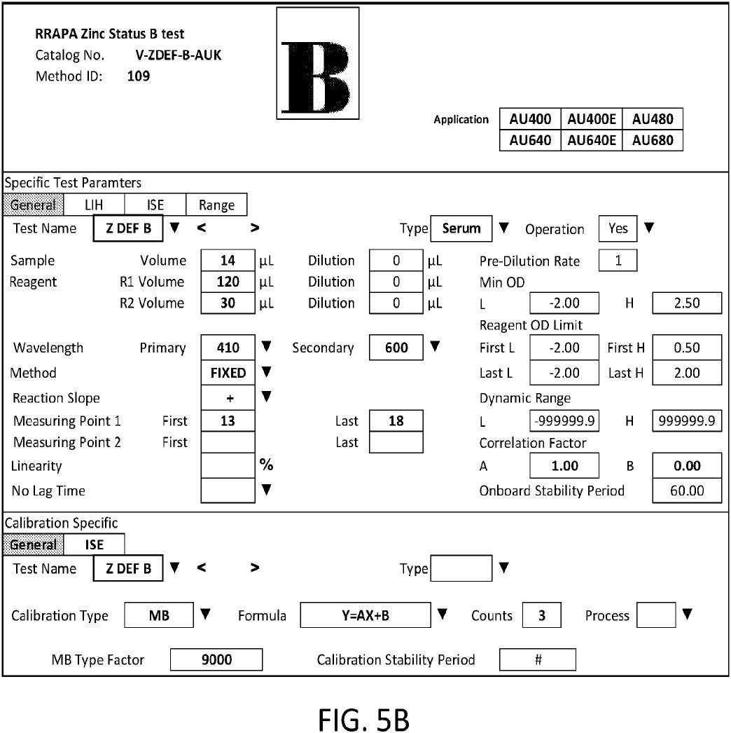

The impractical use of 24 hour collection for the Predicate studies was undertaken because the relative dilution/concentration of single collections is variable over a 24 hour period. Quantitative creatinine tests were performed in parallel with quantification of Alkaline Phosphatase (ALP) using the RRAPA assay as a measure of relative dilution/concentration. The creatinine test that was used had been validated to have linearity to 500 mg/dL. Creatinine was used as an indication of the relative dilution/concentration of each specimen. Graphical analysis of the activity of ALP with the Optimal Zinc Reagent I with the A Assay (FIG. 4A) and the Zinc-Free Reagent II with the B Assay (FIG. 4B).

The graphs indicate that higher specimen concentration represented by higher creatinine levels tend to increase values for alkaline phosphatase in both the A and the B test. Values are generally higher in the A test, but the pattern of increase is similar in both A and B results. However, the lack of a more perfect linear correlation indicates that the amount of urine ALP also varies for reasons other than concentration/dilution.

The use of the amount of renal-derived alkaline phosphatase in the specimen is unlikely to be a valid indicator of zinc status in either a random or 24 hour collection.

For this reason, embodiments of the subject invention utilize a paired zinc-free (B Assay) and zinc-optimal (A Assay) to determine the relative activity as the percentage calculated by the B Assay/A Assay.

### The Effect on RRAPA % Results on 200 Un-Centrifuged Specimens

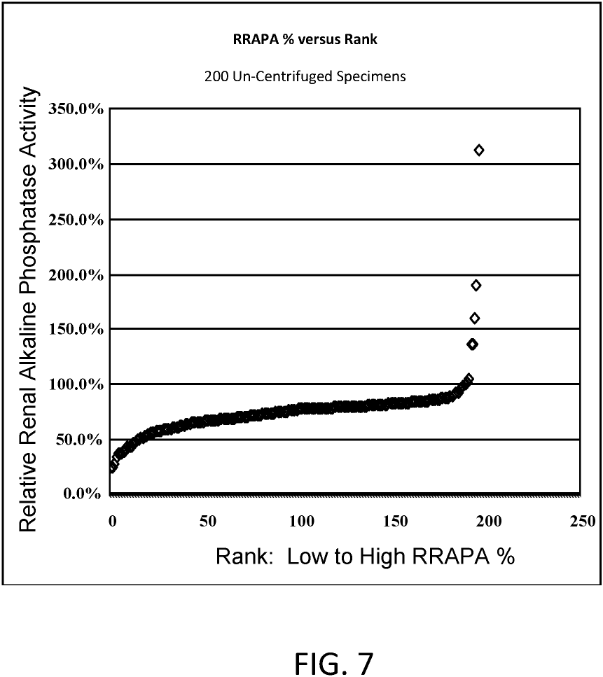

To determine the effect of centrifugation on results of the ALP utilized as the RRAPA test’s biomarker, a percentage activity of B Assay/A Assay results was calculated from the data in the graphs above which is shown in FIG. 7.

The graphical data for non-centrifuged specimens shows five spuriously high values which 4D statistical analysis shows not to be of the same population as the other 195 values. Four of the spurious values were female specimens and one a male specimen. As further elaborated in the Protocol E discussion below, these spurious values are believed to be due to non-renal cellular contamination. The tests utilized in the Predicate studies are prone to zinc biomarker contamination which centrifugation cannot remove.

### RRAPA Analysis of the Supernatant of Centrifuged Specimens

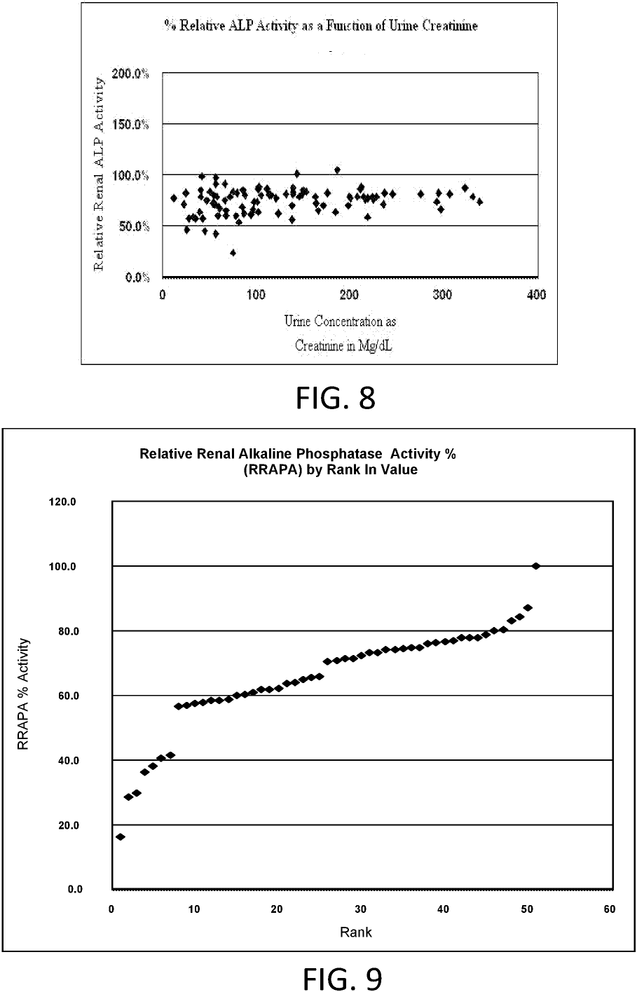

The RRAPA assay biomarker activity results shows little or no effect of urine concentration/ dilution using random urine collection, as shown in FIG. 8.

This verifies that a random urine collection as currently used for routine urinalysis can also be used with embodiments of the RRAPA assay and method for calculating zinc tissue levels of the subject invention, thereby eliminating the need for a 24 hour urine collection.

Protocol B: The embodiments of the RRAPA assay and method for calculating zinc levels status of the subject invention addresses the problem with Predicate studies’ cutoff values with a protocol to establish cutoff values by both statistical and graphical analysis. The graphical analysis is particularly useful as the data does not appear to be Gaussian.

Data from 51 specimens collected for urinalysis were assayed with the RRAPA zinc levels status method after the testing for which they were collected was completed. The age, sex, and collection time information on the specimens was available, but not the patient name. Two laboratory workers provided samples that were included in the test.

In this study, the urine samples were centrifuged to remove cellular contamination and the supernatant was used for the assay. The IFU requires that urine samples showing pink colored supernatants are to be rejected as unsuitable for the RRAPA assay. No specimens were required to be rejected for this study.

D statistical analysis was performed revealing that two populations of data were present at the 95% confidence level. Deviations from the mean of all data were calculated, and those deviating by greater than 2.5 times the average deviation were placed in a separate population. A total of 7 specimens having RRAPA values below 42% were classified as abnormal lows by this means.

The “normal” population was subjected to statistical analysis using Student t test which yielded a mean of 70.5 percent and a low limit of 50.4 and an upper limit of 90% at the 95% confidence level. Graphical representation of the data is shown in FIG. 9.

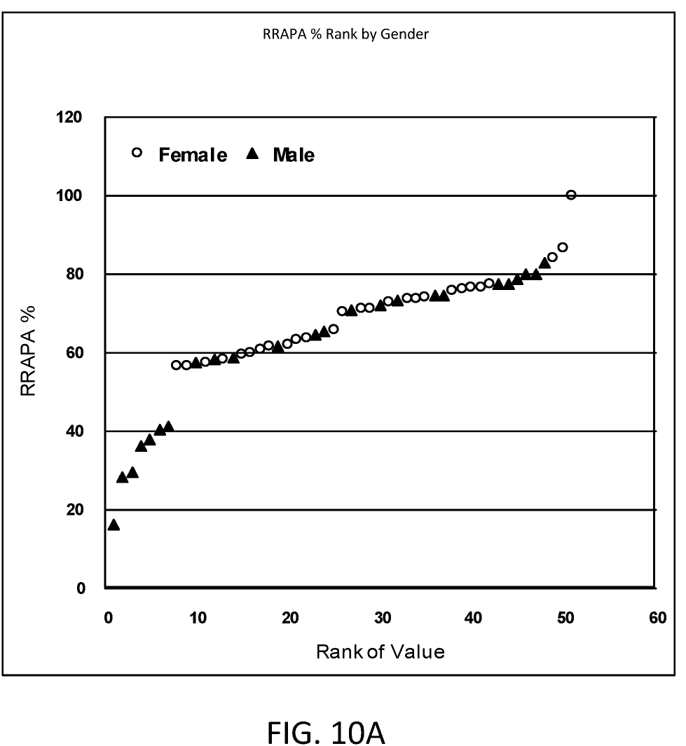

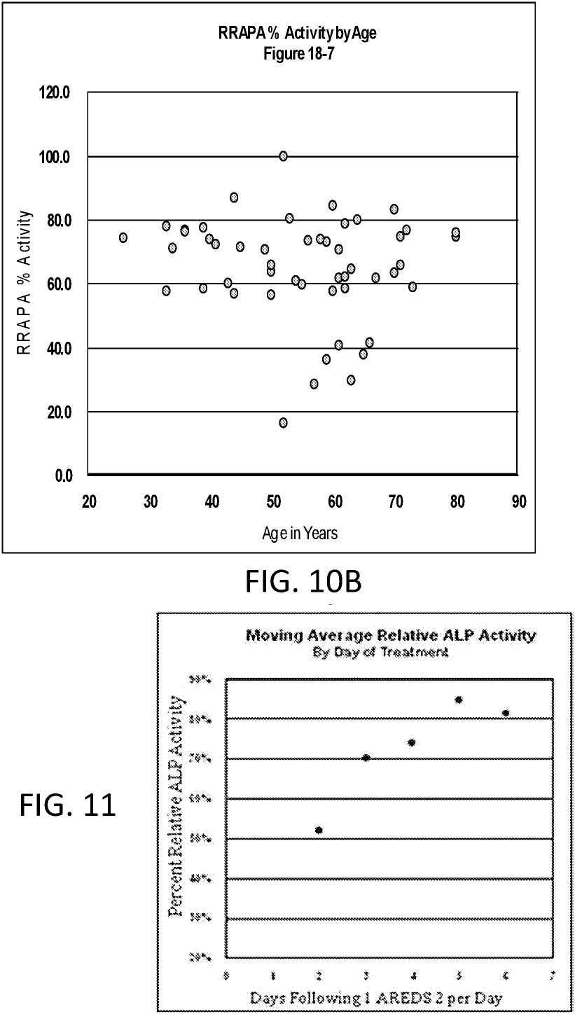

The different populations are apparent upon inspection of the data which appears to confirm the statistical analysis. FIG. 10A graphs values by gender and FIG. 10B graphs values by age.

### Preliminary Bench Testing

All specimens were single random collections. Specimens were stored at between 2-8° C. in refrigerators, which were monitored 24 hours per day in accordance with Validity Diagnostics Quality Management Procedures.

Voluntary specimens were collected by the Applicant, Validity Diagnostics, Inc. and affiliate company North Florida Biomedical, Inc. technical laboratory staff.

Additionally, eighteen people from a church in Branford, FL each volunteered to donate a urine specimen for this test. The average age was 72.5 years with a range of 53-87 years. All completed a questionnaire indicating whether they:
regularly took a vitamin/mineral or other supplement-27.8%;
were under treatment for high blood pressure including diuretics such as Lasix -16.7%;
were on a vegetarian or vegan diet - 0%; and/or
were diagnosed as diabetic - 16.7% (Described as type II).

Two of eighteen subjects tested as zinc deficient and 1 subject was considered borderline deficient. All were female.

Most zinc deficient subject A
Age 88;
Urine RRAPA 26.8%;
Interview indicated she had consumed only distilled drinking water over decades;
No zinc or mineral supplement intake;
Severe lordosis of the spine associated with arthritis symptoms; and/or
Treated for high blood pressure.

Deficient subject B
Age 79;
Urine RRAPA 49.5%;
No zinc or mineral supplement intake;
Treated for high blood pressure including furosemide diuretics for several years; and/or
Diabetes II.

Borderline deficient subject C
Age 52;
Urine RRAPA 61.5%;
No zinc or mineral supplement intake;
Treated for high blood pressure including Lasik type diuretic for past year;
Diabetes II; and/or
Scoliosis of the spine.

Patients chronically taking Lasik type diuretics and ACE inhibitors are known to have tissue zinc deficiency at autopsy while having elevated urine zinc levels. The RRAPA biomarker findings of this study are consistent with tissue levels, conversely the zinc biomarker methods described in the Predicate studies would be likely to give a false high assessment of tissue zinc status.

### Assay Modification Based on Preliminary Testing

Reagent formulae for the Subject Device were not changed during all of the testing and the reagent composition of embodiments of the RRAPA of the subject invention is the same as used for testing.

Urine Sample volume was decreased to 14 µL from 25 µL making the urine sample to RRAPA two paired reagents’ ratios about 1:10.7. This change was made because it gave greater separation between low and high RRAPA values. The improved detection obtained by utilization of the smaller urine sample volume is believed to be due to less activation of Renal Alkaline Phosphatase by urine zinc. It is known that urine zinc is elevated by antihypertensive drugs such as Lasix and Furosemide, while tissue levels are zinc deficient. The preliminary Bench testing results indicate that the true renal tissue levels were detected more accurately at the 14 µL sample volume level. The final sample volume chosen was based upon optimizing between tissue specificity and precision of results.

Protocol C: Is designed to measure the RRAPA assay biomarker response after controlled manipulations of zinc intake, including both zinc-depletion/replication studies and zinc supplementation.

Case Study 1: Subject A above, with the physician permission, volunteered to be tested following the daily ingestion of one (1) Bausch & Lomb PreserVision^{®} AREDS 2 containing 45 mg of zinc per day. (Note: the recommended dosage indicated on the label is 2 per day). The result was a progressive increase in urine relative-activity as shown in Table 2 and FIG. 11.

TABLE 2:

| TREATMENT DAY | RELATIVE ACTIVITY | MOVING AVERAGE |
|---|---|---|
| 0 | 29.6% | 29.6% |
| 2 | 74.4% | 52.0% |
| 3 | 88.7% | 70.3% |
| 4 | 77.5% | 73.9% |
| 5 | 95.6% | 84.7% |
| 6 | 78.4% | 81.6% |

Case study 2: A male, 50 years of age volunteered to be part of this study. He confirmed having taken Vitamin D3 as his only medication. He began taking One (1) AREDS 2 supplement (40 mg), which raised urine RRAPA biomarker levels from low normal to a mid-normal level. He then increased to 2 AREDS-2 per day (80 mg) and reached optimal Relative ALP activity levels.

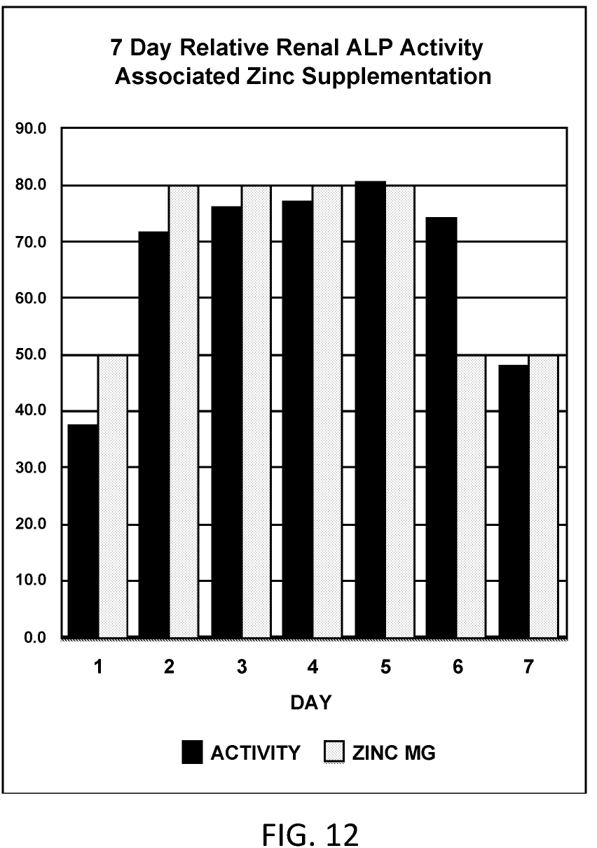

He then volunteered to return to 1 per day. FIG. 12 provides the results of these controlled manipulations of zinc intake, including both zinc-depletion/replication studies and zinc supplementation. In FIG. 12, the Y-axis is both the RRAPA biomarker percent activity and mg of zinc supplement, respectively.

The RRAPA biomarker showed a rapid increase on the first day followed by a progressive increase to 80% relative activity. On day 6, the subject returned to the 50 mg zinc supplement which resulted in a small initial decrease in RRAPA biomarker followed by a larger decrease on day 7. The RRAPA biomarker had not returned completely to pre-zinc increase levels but was trending to those levels. Both the progressive increase and progressive decrease RRAPA biomarker reflects tissue levels of zinc.

Case study 3: In another study with the same male subject as in Case Study 2, a store brand equivalent for PreserVision^{®} was used. PreserVision® also contains zinc. The return to optimal RRAPA biomarker levels occurred significantly less rapidly that with the Areds-2 product. The zinc in AREDS 2 is believed to be zinc picolinate whereas the zinc in the store brand PreserVision® is zinc oxide, which is less soluble.

It was concluded that the RRAPA two paired reagents and method obtained a biomarker response after controlled manipulations of zinc intake, including both zinc-depletion/replication studies and zinc supplementation, which meets the standard of NIH experts for effective detection of zinc level status.

Protocol D: This study was designed to compare the RRAPA reagent and method for determining the biomarker level between individuals by using clinical signs that are generally recognized as functional outcomes of severe zinc deficiency as recommended by the NIH Expert Panel.

A 1980 report stated that urinary zinc levels were elevated by diuretics often used to treat hypertension while tissue levels of zinc in 147 autopsies revealed that a lower tissue zinc was observed in patients who had been on diuretic treatment for more than 6 months (Wester, P.O., Tissue zinc at autopsy - relation to medication with diuretics, Acta Med Scand, 1980;208(4): 269-71).

A 2006 report states that hypertension may enhance zinc deficiency. Thiazides may cause a decrease in tissue zinc levels. Furosemid patients with chronic furosemide treatment showed low tissue zinc levels at autopsy. Treatment with angiotensin converting enzyme (ACE) inhibitors and angiotensin receptor blockers (ARBs) have produced zinc deficiencies in some studies (Nathan Cohen, Ahuva Golik, Zinc Balance and Medications Commonly Used in the Management of Heart Failure, Heart Fail Rev. 2006 Mar; 11(1):19-24 https//pubmed.nlm.nih.gov/16819574/).

Three of the eighteen volunteers from the Church in Branford, FL, mentioned above, tested with the RRAPAtwo paired reagents and method were found to have a deficient tissue zinc status. All three had clinical signs or known causes of zinc deficiency.

Subject A presented as the most deficient volunteer:
Age 88;
Urine Relative ALP Activity 26.8%;
Interview indicated she had consumed only distilled water for drinking over decades;
No zinc or mineral supplement intake;
Severe lordosis of the spine associated with arthritis symptoms; and/or
Treated for high blood-pressure.

The long-term lack of dietary or supplement zinc intake, the lordosis/arthritic syndrome and potential use of diuretic for blood pressure treatment are clinical signs or known causes of zinc deficiency. The fact that zinc supplementation restored Relative renal ALP activity to near optimal levels is further confirmation.

Subject B also exhibited signs of zinc deficiency:
Age 79;
Urine ALP relative activity 49.5%;
No zinc or mineral supplement intake;
Treated for high blood pressure including furosemide diuretics for several years; and/or
Diabetes II.
The chronic use of furosemide and Diabetes are clinical signs or known causes of zinc deficiency.

Subject C exhibited borderline zinc deficiency:
Age 52;
Urine ALP relative activity 61.5%;
No zinc or mineral supplement intake;
Treated for high blood pressure including Lasik type diuretic for past year;
Diabetes II; and/or
Scoliosis of the spine.
The use of Lasik type diuretic, Diabetes and Scoliosis of the spine and are clinical signs or known causes of zinc deficiency.

It was concluded that the RRAPA two paired reagents and method meet the criteria of the Expert Panel that detection is related to clinical signs that are generally recognized as functional outcomes of severe zinc deficiency.

### Protocol E - Specimen Contamination With Biomarker

The Design Control process used in this study requires that risks that may be associated with similar studies must also be considered as potential risks for this study. The previous studies that utilized zinc concentration in serum, plasma, or urine as an indication of zinc status is known to have risks associated due to the presence of elevated levels of the zinc biomarker in urine. These elevated levels can be caused by factors other than tissue zinc status, which confound results because the urine zinc level is caused these factors to be opposite of the tissue zinc status thereby masking the true zinc status.

For example, certain drugs complex plasma/serum zinc removing the zinc from proteins. Most serum zinc is associated with serum proteins and therefore the protein associated zinc does pass through the glomerular filtration process and enter the urine. However, the drug associated zinc does pass through the glomerular filtration into the urine raising zinc levels. The principal drugs involved are ACE inhibitors, beta blockers, and diuretics used to control hypertension. These same drugs are known to lower tissue zinc levels.

Contamination of the 24-hour urine collections required by the Predicate studies that measured zinc concentration in serum, plasma, or urine as an indication of zinc status are known to make contamination with zinc a risk which can mask low tissue zinc status.

The detection of expectedly low tissue levels by the RRAPA two paired reagents and method indicates that the masking risk of the Predicate studies is not a risk with the RRAPA reagent and method utilized with this study.

Unlike the zinc biomarker of the Predicate studies the embodiments of the Relative Alkaline Phosphatase Activity RRAPA biomarker and method is not ubiquitously present in dust and other environmental contributors. However, non-renal alkaline phosphatase is present in vaginal epithelial cells, erythrocytes, leucocytes, trichomonas vaginalis, and other non-renal cellular elements. Erythrocytes are a particular concern as erythrocytes contain 10X the level of zinc compared to plasma. This means that the erythrocyte alkaline phosphatase should have a high relative activity which could mask abnormally low RRAPA levels.

It has been determined by the inventor that centrifugation of the urine for assay with the RRAPA reagents and method of the subject invention prior to analysis can remove cellular elements, which might otherwise mask abnormal low values. In contrast, the Predicate studies that measure zinc concentration cannot remove interference from non-tissue related zinc biomarker by centrifugation.

The effectiveness of the risk elimination by centrifugation of specimens was tested by adding one drop of fresh blood to 50 mL of urine collected from two female laboratory workers. One of the workers was known to regularly produce very dilute urine.

### Results

Female A: Urine Creatinine Normal

The non-centrifuged specimen was only slightly lower (5.7 %) than the centrifuged urine. The presence of blood in the specimen severely lowered both the A and B Assay results, which gave an abnormal low RRAPA value. Analysis of the supernatant centrifuged blood containing specimen produced a RRAPA value which was nearly identical to the supernatant before addition of blood.

The addition of baker’s yeast significantly decreased (11.6%) the RRAPA value in the non-centrifuged specimen compared to the non-yeast containing centrifuged sample. Centrifugation of the yeast containing specimens produced RRAPA values approximating centrifuged specimen containing no yeast. The RRAPA values for both blood and yeast containing specimens stored refrigerated at 2 - 8° C. for 2 and 3 days did not change significantly indicating that the centrifugation step may take place following sample transport. Table 3 summarizing the results obtained from Female A urine analysis:

TABLE 3

FEMALE A SPECIMEN: FRESH URINE

| SAMPLE | A | B | RRAPA % |
|---|---|---|---|
| SR Non Centrifuged | 89.6 | 64.3 | 71.8 |
| SR Centrifuged | 86.9 | 66.1 | 76.1 |
| SR Non Centrifuged Blood | -14.8 | -2.2 | 14.9 |
| SR Centrifuged Blood | 87.8 | 66.1 | 75.3 |
| SR Non Centrifuged Yeast | 101.3 | 67.9 | 67.0 |
| SR Centrifuged Yeast | 86 | 65.2 | 75.8 |
| Urine Creatinine = 55 mg/dL | | | |
| FEMALE A SPECIMEN: 2 DAYS REFRIGERATED | | | |
| SAMPLE | ZDEF A | ZDEF B | ZDEF % |
| SR Non Centrifuged Blood | -19.3 | -4 | 20.7 |
| SR Centrifuged Blood | 86 | 68.9 | 80.1 |
| SR Non Centrifuged Yeast | 92.3 | 62.5 | 67.7 |
| SR Centrifuged Yeast | 86.9 | 68 | 78.3 |
| FEMALE A SPECIMEN: 3 DAYS REFRIGERATED | | | |
| SAMPLE | ZDEF A | ZDEF B | ZDEF % |
| SR Non Centrifuged Blood | -15.7 | -3.1 | 19.7 |
| SR Centrifuged Blood | 87.8 | 66.1 | 75.3 |
| SR Non Centrifuged Yeast | 95.9 | 67.1 | 70.0 |
| SR Centrifuged Yeast | 87.8 | 66.1 | 75.3 |

Female B: Urine creatinine very low indicating maximal dilution.

TABLE 4

FEMALE B SPECIMEN: FRESH URINE

| SAMPLE | A | B | RRAPA % |
|---|---|---|---|
| * SUZ Centrifuged | 16.7 | 21.2 | 126.9 |
| SUZ Non Centrifuged Blood | -34.6 | -23.8 | 68.8 |
| SUZ Centrifuged Blood | 1.4 | 8.5 | 607.1 |
| SUZ Non Centrifuged Yeast | 32 | 25.6 | 80 |
| SUZ Centrifuged Yeast | 16.7 | 23 | 137.7 |

Urine Creatinine = 23.8 mg/dL
*Supernatent was visbly pink

The osmotic pressure caused by the extreme dilution of the urine caused lysis of some or all of the erythrocytes, which was indicated by a pink/red color of the supernatant following centrifugation. The hemolysis in the supernatant greatly increased the RRAPA compared to the blood free specimen, which invalidated the test. Therefore, it would be beneficial for the Instructions for Use (IFU) for the RRAPA assay and method of the subject invention to instruct the analyst to inspect the centrifuged specimens for pink color and, if detected, to report invalid specimen due to hemolysis.

### Conclusions

Centrifugation of samples can be effective in eliminating contaminating biomarker that can affect results when utilizing embodiments of the RRAPAtwo paired reagents and method, provided that the supernatant is inspected for pink/red color and such samples are reported as unsuitable for analysis.

The elimination of biomarker contamination combines with true detection of tissue zinc induced by certain drugs or physiologic conditions. The Predicate studies can give a false indication because certain drugs increase the urine zinc biomarker of the Predicate studies. Therefore, the RRAPA system introduces minimal or no new risk and eliminates known risks associated with the Predicate studies.

### EXAMPLE 4: Correlation of RRAPA Value With Zinc Status and Severity of Symptoms Related to Covid-19 Infection

Twenty-three subjects were studied for 12 months beginning in March of 2020 and ending in March of 2021. The supplement history was as follows:

| | |
|---|---|
| Full Dose - 2 AREDS 2 per day + 1,000 units vitamin D3 | 16 people |
| Half dose - 1 AREDS 2 per day + 1,000 units vitamin D3 | 2 persons |
| No supplement | 5 people |

None of the people taking a Full Dose of AREDS 2 reported symptomatic Covid-19 infection. Of those taking ½ dose, one had no symptoms the other had moderate Covid-19 symptoms requiring hospitalization and oxygen treatment. Both were females. The female with no symptoms was 87 years old and very thin. Her RRAPA value tested at 90+ percent. It is speculated that her body mass was low so that ½ dose was more effective. The lady who had moderate Covid-19 symptoms was somewhat overweight and had a near average RAPPA level.

All of those taking no supplement developed symptomatic Covid-19 some requiring Hospitalization.

In addition, seven families and other individuals were included in the survey. These families and individuals were acquainted with the 23 members discussed above. As the people were in various States and other Cities within Florida it was not possible to test all of these additional survey individuals. The use of families was useful since if one member was infected it indicated that the others were most likely exposed. The details of each family group and the individuals are summarized in the following Tables 5 and 6.

TABLE 5

Family Survey

| Q.S. Member | State | Gender | Age | 2 AREDS + D 3 | RRAPA Tested? | Risk Factor | Known Exposure | COVID OUTCOME |
|---|---|---|---|---|---|---|---|---|
| R.B | MO | M | 65 + | NO | NO | Age | Yes | Severe Covid 19 with hospitalization |
| R.B | MO | F | 65 + | 2 AREDS + D3 | NO | Age | Yes | Asymptomatic |
| R.B | FL | M | 65 + | 2 AREDS + D3 | 100% | Age | Yes | Asymptomatic |
| R.B | FL | F | 60 | 2 AREDS + D3 | NO | Age | Yes | Asymptomatic |
| R.B | FL | M | 25 | NO | NO | None | Yes | Covid 19 2 week recovery No hospitalization |
| R.B | FL | F | 20 | 2 AREDS + D3 | NO | None | Yes | Asymptomatic |
| SR | FL | M | 75 + | 2 AREDS + D3 | 90% | Age | Yes | Asymptomatic |
| SR | FL | F | 75 + | 2 AREDS + D3 | 90% | Age | Yes | Asymptomatic |
| JD | NC | M | 65 + | NO | NO | Age | Yes | Covid 19 2 week recovery No hospitalization |
| JD | NC | F | 60 | 2 AREDS + C3 | NO | Exposure | Yes | Asymptomatic |
| KM | FL | M | 60 | 2 AREDS + C3 | 90% | Age | Yes | Asymptomatic |
| KM | FL | F | 60 | 2 AREDS + C3 | 90% | Age | Yes | Asymptomatic |
| KM | FL | F | 80+ | 2 AREDS + C3 | NO | Age + Asthma | Yes | Asymptomatic |
| KM | FL | F | 80+ | 2 AREDS + C3 | NO | Age Hypertension | ? | Asymptomatic |
| KM | FL | M | 50 | NO | 60% | Low Zn | Yes | Covid 19 2 week recovery No hospitalization |
| KM | FL | F | 50 | NO | NO | Low Zn | Yes | Covid 19 2 week recovery No hospitalization |
| JD | FL | M | 55 | 2 AREDS + D3 | NO | Age | ? | Asymptomatic |
| JD | FL | F | 50 | 2 AREDS + D3 | NO | Age | ? | Asymptomatic |

TABLE 6

Individuals

| Q. S. Member | State | Gender | Age | 2 AREDS + D 3 | RRAPA Tested? | Risk Factor | Known Exposure | COVID OUTCOME |
|---|---|---|---|---|---|---|---|---|
| SR | IN | F | 50 | 2 AREDS + D3 | NO | Diabetic Overweight | Yes | Asymptomatic |
| SR | IN | F | 66 | 2 AREDS + D3 | NO | Age | Yes | Asymptomatic |
| JD | IN | F | 65 | ½ Dose | 63% | Age + Overweight Postsurgical | Yes | Infected, No Fever, Low Oxygen Hospitalized 4 days on oxgen, Recovered |
| JD | FL | F | 80+ | ½ Dose | 90% | Age | No | Asymptomatic |

Thus, 73.9 % of individuals taking a Full of a Half Dose of AREDS 2 were Covid-19 symptom free. The individuals that did not take any AREDS 2 had symptomatic Covid-19.

### CONCLUSION

This preliminary survey was limited by the number of people included and many of the RRAPA percentages had to be inferred for the subjects who were not tested.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

The examples and embodiments described herein are for illustrative purposes only and that various modifications or changes in light thereof will be suggested to persons skilled in the art and are to be included within the spirit and purview of this application.

I claim:

1. A kit, for obtaining a Relative Renal Alkaline Phosphatase Activity (RRAPA) value for a urine sample, comprising:
a Reagent I comprising,
a non-reactive sub-reagent comprising zinc sulfate,
a reactive sub-reagent comprising N-nitrophenylphosphate;
a Reagent II comprising,
a non-reactive sub-reagent having a composition identical to the non-reactive sub-reagent of Reagent I, without the zinc sulfate; and
a reactive sub-reagent having a composition identical to the reactive sub-reagent of Reagent I,
wherein, the results obtained from Reagent I and the results obtained with Reagent II are utilized in a RRAPA Equation to obtain the RRAPA value.

2. The kit according to claim 1, wherein Reagent I and Reagent II further comprise a detergent.

3. The kit according to claim 2, wherein the detergent comprises at least one of Brij 35 and Triton-X 100.

4. The kit according to claim 3, wherein Reagent I and Reagent II further comprise a preservative.

5. The kit according to claim 3, wherein Reagent I and Reagent II are configured for use with an automated clinical chemistry analyzer.

6. A method for utilizing an assay to obtain a Relative Renal Alkaline Phosphatase Activity (RRAPA) value for a urine sample, the method comprising:
obtaining a kit, according to claim 1,
conducting an A Assay comprising,
combining a first portion of the urine sample with the non-reactive sub-reagent of Reagent I,
waiting a pre-determined time,
adding further the reactive sub-reagent of Reagent 1 to the first portion of the urine,
measuring spectrophotometrically an amount of N-nitrophenol formed after adding the reactive sub-reagent;
conducting a B Assay comprising,
combining a second portion of the urine sample with the non-reactive sub-reagent of Reagent II,
waiting a pre-determined time,
adding further the reactive sub-reagent of Reagent II to the second portion of the urine
measuring spectrophotometrically the N-nitrophenol formed after adding the reactive sub-reagent;
utilizing the results of the A Assay and the B Assay in the following equation:
RRAPA value= (Result of the B Assay/Result of the A Assay) * 100,
wherein the RRAPA value corresponds to the activity of alkaline phosphatase in the urine sample.

7. The method according to claim 6, further comprising centrifuging the urine sample to obtain a supernatant and utilizing a first portion of the supernatant for conducting the A Assay and utilizing a second portion of the supernatant to form the B Assay.

8. The method according to claim 7, wherein the first portion of supernatant comprises about 9% of a total volume of Reagent I.

9. The method according to claim 8, wherein the second portion of supernatant comprises about 9% of the total volume of Reagent II.

10. The method according to claim 6, further comprising utilizing a weighting factor with the measurement obtained with the A Assay.

11. The method according to claim 10, wherein the weight factor is between about 3, 4, and 5.

12. The method according to claim 11, wherein the A Assay and the B Assay are performed with an automated clinical chemistry analyzer programmed to calculate the RRAPA value.

13. The method according to claim 12, further comprising programming the automated clinical chemistry analyzer with a numeric factor.

14. The method according to claim 13, wherein the RRAPA value is correlated to a zinc level in renal tissue.

15. A kit, for obtaining a Relative Zinc-Activated Enzyme Activity (RZAEA) value for a zinc-activated tissue enzyme in a bodily fluid sample, comprising:
a Reagent I comprising a composition that includes zinc for measuring the total activity of the zinc-activated enzyme in the bodily fluid sample; and
a Reagent II comprising a composition that is identical to the composition of Reagent I, but without zinc, for measuring the native-activity of the zinc-activated enzyme in the bodily fluid sample.

16. The kit according to claim 15, wherein the results obtained from Reagent I and the results obtained with Reagent II are utilized in a RZAEA equation to obtain the RZAEA value.

17. The kit according to claim 16, wherein the RZAEA value is correlated to a zinc level in a body tissue.

18. The kit according to claim 15, wherein Reagent I further comprises a non-reactive sub-reagent and a reactive sub-reagent, and wherein Reagent II further comprises a non-reactive sub-reagent and a reactive sub-reagent.

19. A method for utilizing an assay to obtain a Relative Zinc-Activated Enzyme Activity (RZAEA) value for a bodily fluid sample, the method comprising:
obtaining a kit according to claim 15,
conducting an A Assay comprising,
combining a first portion of the bodily fluid sample with Reagent I,
measuring spectrophotometrically a product formed by the zinc-activated enzyme, after a pre-determined period;
conducting a B Assay comprising,
combining a second portion of the bodily fluid sample with Reagent II;
measuring spectrophotometrically the product formed by the zinc-activated enzyme, after a pre-determined period;
utilizing the results of the A Assay and the B Assay in the following equation:
RZAEA value= (Result of the B Assay/Result of the A Assay) * 100,
wherein the RZAEA value corresponds to the activity of the zinc-activated enzyme in the bodily fluid sample.

20. The method according to claim 19, wherein Reagent I further comprises a non-reactive sub-reagent and a reactive sub-reagent, and the method further comprises adding the non-reactive sub-reagent to the bodily fluid sample and subsequently adding the reactive sub-reagent to initiate formation of the product.

21. The method according to claim 20, wherein Reagent II further comprises a non-reactive sub-reagent and a reactive sub-reagent, and the method further comprises adding the non-reactive sub-reagent to the bodily fluid sample and subsequently adding the reactive sub-reagent to initiate formation of the product.

22. The method according to claim 19, wherein the A Assay and the B Assay are performed with an automated clinical chemistry analyzer programmed to calculate the RZAEA value.

23. The method according to claim 22, wherein the RZAEA value is correlated to a zinc level in tissue.
